SEQUENCE LISTING (20 sequences)

SEQ ID NO: 1 - DNA, 363 bp, Artificial
  ANTIBODY
caggtgcagctgcaggagtcaggacctgacctggtgaagcctggggcttcagtgaagatgtcctgcaaggcttctggttactcattcacttactaccacatgcactgggtaaagcagacccatggaaagagccttgagtggattggacgtatttatccttacaatggtggcactaactacagccagaggttcaagggcaaggccgtatttactgtggacatgtcatccagcacagcctacatggagctccgcagcctgacatctgaggactctgcggtctattactgtgccagagatgctaactacgagggggactggtacttcgatgtctggggccaagggaccacggtcaccgtctcctca

SEQ ID NO: 2 - DNA, 321 bp, Artificial
  ANTIBODY
gacatccagatgacgcagtctccatcctccttttctgtatctctaggagacagagtcaccattacttgcaaggcaagtgaggacatatataatcggttagcctggtatcagcagaaaccaggaaatgctcctaggctcttaatatctgatacaaccaatttgtggattggggttccttcaagattcagtggcagtggatctggaaaggattacactctcagcattaccagtcttcagagtgaagatgttgctacttattactgtcaacagtattggagttctcggacgttcggtggaggcaccaagctggaaatcaaacgg

SEQ ID NO: 3 - DNA, 363 bp, Artificial
  ANTIBODY
caggtgcagctgcaggagtctggacctgacctggtgaagcctggggcttcagtgatgatatcctgcaaggcttctggttactcattcacttactactacatgcactgggtgaagcagagccatggaaagacccttgagtggattggacgtatttatccttacaatggtgatactaactacaaccggaggttcaaggacaaggccatattaactgtagacgagtcatccagcacagcctacatggagctccgcagcctgacatctgaggactctgcggtctatttctgtgcaagagatgctaactacgagggggactggtacttcgatgtctggggccaagggaccacggtcaccgtctcctca

SEQ ID NO: 4 - DNA, 321 bp, Artificial
  ANTIBODY
gacatccagatgactcagtctccatcctccttttctgtatctctgggagacagagtcaccattacttgtaaggcaagtgaggacatatataatcgggtagcctggtatcagcagaaaccaggaaatgctcctaggctcttaatgtctgatgcaaccagtttgggagctggggttccttcaagattcagtggcagtggatctggaaaggatcacactctcagcattaccagtcttcagactgaagatgttgctacttattactgtcaacaatattggaatactcggacgttcggtggaggcaccaagctggaaatcaaacgg

SEQ ID NO: 5 - protein, 120 aa, Artificial
  ANTIBODY
QVQLQESGPDLVKPGASVKMSCKASGYSFTYYHMHWVKQTHGKSLEWIGRIPYNGGTNYSQRFKGKAVFTVDMSSSTAYMELRSLTSEDSAVYYCARDANYEGDWYFDVWGQGTTVTVSS

SEQ ID NO: 6 - protein, 107 aa, Artificial
  ANTIBODY
DIQMTQSPSSFSVSLGDRVTITCKASEDIYNRLAWYQQKPGNAPRLLISDTTNLWIGVPSRFSGSGSGKDYTLSITSLQSEDVATYYCQQYWSSRTFGGGTKLEIKR

SEQ ID NO: 7 - protein, 121 aa, Artificial
  ANTIBODY
QVQLQESGPDLVKPGASVMISCKASGYSFTYYYMHWVKQSHGKTLEWIGRIYPYNGDTNYNRRFKDKAILTVDESSSTAYMELRSLTSEDSAVYFCARDANYEGDWYFDVWGQGTTVTVSS

SEQ ID NO: 8 - protein, 107 aa, Artificial
  ANTIBODY
DIQMTQSPSSFSVSLGDRVTITCKASEDIYNRVAWYQQKPGNAPRLLMSDATSLGAGVPSRFSGSGSGKDHTLSITSLQTEDVATYYCQQYWNTRTFGGGTKLEIKR

SEQ ID NO: 9 - protein, 552 aa, Influenza A virus
DQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 10 - protein, 552 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPANDLCYPGDFNDYEELKHLLSRINHFEKIQIIPKNSWSSHEASLGVSSACPYQGKSSFFRNVVWLIKKNNAYPTIKRSYNNTNQEDLLVLWGIHHPNDAAEQTRLYQNPTTYISVGTSTLNQRLVPKIATRSKVNGQNGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 11 - protein, 551 aa, Influenza A virus
DQICIGYHANNSTEQVDTIMEKNVTVTHAQDILEKTHNGKLCDLDGVKPLILRDCSVAGWLLGNPMCDEFINVPEWSYMVEKANPANDLCYPGNFNDYEELKHLLSRINHFEKIQIIPKSSWSDHEASLGVSSACPYQGSSSFFRNVVWLIKKNNAYPTIKRSYNNTNQEDLLVLWGIHHPNDEAEQTRLYQNPTTYISVGTSTLNQRLIPKIATRSKVNGQSGRMDFFWTILKPNDAINFESNGNFIAPEYAYKIVKKGDSAIMKSEVEYGNCNTKCQTPMGAINSSMPFHNIHPLTIGECPKYVKSNKLVLATGLRNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 12 - protein, 552 aa, Influenza A virus
DQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 13 - protein, 551 aa, Influenza A virus
DQICIGYHANNSTEQVDTIMEKNVTVTHAQDILEKTHNGKLCDLDGVKPLILKDCSVAGWLLGNPMCDEFINVPEWSYIVEKANPAYDLCYPGNFNDYEELKHLLSRINHFEKIQIIPKSSWSDHEASLGVSSACPYQGNSSFFRNVVWLIKKNDAYPTIKRSYNNTNQEDLLVLWGIHHPNDEAEQTRLYQNPTTYVSIGTSTLNQRLVPKIATRSKVNGQSGRIDFFWTILKPNDAINFESNGNFIAPEYAYKIVKKGDSAIMKSEVEYGNCNTKCQTPMGAINSSMPFHNIHPLTIGECPKYVKSNKLVLATGLRNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLVLAIMVAGLSLWMCSNGSLQCRICI

SEQ ID NO: 14 - protein, 552 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGNPSFFRNVVWLIKKNSTYPTIKRNYNNTNQEDLLVMWGIHHPNDAAEQTKLYQNPTTYISVGTSTLNQRLVPRIATRSKINGQSGRIEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 15 - protein, 552 aa, Influenza A virus
DQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPHQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 16 - protein, 411 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGTPSFFRNVVWLIKKNNTYPTIKRSYNNTNQEDLLILWGIHHSNDAAEQTKLYQNPTTYISVGTSTLNQRLVPKIATRSKVNGQSGRMDFFWTILKPNDAINFESNGNFIAPEYAYKIVKKGDSAIMKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLIK

SEQ ID NO: 17 - protein, 552 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPANGLCYPGNFNDYEELKHLLSRINHFEKLQIIPKSSWSDHEASLGVSSACPYLGRSSFFRNVVWLIKKNNTYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGDCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGIYQILSIYSTVASSLALAIMMAGLSLWMCSNGSLQCRVCI

SEQ ID NO: 18 - protein, 552 aa, Influenza A virus
DQICIGYHANNSTEQVDTIMEKNVTVTHAQDILEKTHNGKLCDLDGVKPLILRDCSVAGWLLGNPMCDEFINVPEWSYIVEKASPANDLCYPGDFNDYEELKHLLSRINHFEKIQIIPKSSWSNHEASSGVSSACPYLGKPSFFRNVVWLIKKNSAYPTIKRSYNNTNQEDLLVLWGIHHPNDAAEQTKLYQNPTTYISVGTSTLNQRLVPKIATRSKVNGQSGRMEFFWTILKPNDSINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 19 - protein, 552 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPANDLCYPGDFNDYEELKHLLSRINHFEKIQIIPKSSWSNHESSSGVSSACPYLGKPSFFRNVVWLIKKNNAYPTIKKSYNNTNQEDLLVLWGIHHPNDATEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVRNLYDKVRLQLRDNAKELGNGCFEFYHKCDDECMESVRNGTYDYPQYSEEARLKREEISGVKLESIGTYQILSIYSTVASSLALAIMVAGLSLWMCSNGSLQCRICI

SEQ ID NO: 20 - protein, 552 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSYWSDHEASSGVSSACSYLGDPSFFRNVVWLIKKNSAYPTIKRSYNNTNQEDLLVLWGIHHPNDEAEQTRLYQNPTTYISVGTSTLNQRLVPKITTRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPHYSEEARLKREEISGVKLESIGTYQILSIYSTVASSLALAIMMAGLSLWMCSNGSLQCRICI

(19) United States
(12) Patent Application Publication
(10) Pub. No.: US 2010/0150941 A1
(43) Pub. Date: Jun. 17, 2010

(54) HEMAGGLUTININ ANTIBODY AND USES THEREOF

(75) Inventors: Brendon J. Hanson, Singapore (SG); Angeline P. Lim, Singapore (SG); Eng Eong Ooi, Singapore (SG); Adrianus C.M. Boon, Memphis, TN (US); Richard J. Webby, Memphis, TN (US)

Correspondence Address: ALSTON AND BIRD LLP;ST. JUDE CHILDREN'S RESEARCH HOSPITAL, BANK OF AMERICA PLAZA, 101 SOUTH TRYON STREET, SUITE 4000, CHARLOTTE, NC, 28280-4000, US

(73) Assignees: DSO National Laboratories, Singapore (SG); St. Jude Children's Research Hospital, Memphis, TN (US)

(21) Appl. No.: 12/440,113

(22) PCT Filed: Sep. 13, 2007
(86) PCT No.: PCT/SG2007/000310

§ 371 Date: Mar. 1, 2010

Related U.S. Application Data
(60) Provisional application No. 60/844,246, filed on Sep. 13, 2006.

(51) Int. Cl. A61K 39/42; C07K 16/08; C07H 21/00; C12Q 1/70; A61P 31/16

(52) U.S. Cl. 424/159.1; 530/387.1; 530/387.3; 536/23.53; 435/5

(57) ABSTRACT

Passive antibody therapy as a tool for both prophylaxis against—and treatment of—highly pathogenic H5N1 influenza virus, providing immediate immunity is described. It is provided by an antibody specific to hemagglutinin capable of neutralizing influenza viruses and methods of making and using the same, the methods and compounds described herein may be used in diagnostic, prophylactic and therapeutic methods.

### FIELD OF THE INVENTION

The invention relates to methods of making hemagglutinin antibodies and uses thereof including prophylaxis and treatment of influenza.

### BACKGROUND

Highly pathogenic avian H5N1 influenza virus is a major public health concern. Given the lack of effective vaccine and recent evidence of antiviral drug resistance in some isolates, alternative strategies for containment of a possible future pandemic are needed

With the initial outbreak of pneumonia caused by highly pathogenic H5N1 influenza A virus in Hong Kong in 1997 resulting in the death of 6 of the 18 infected individuals (Claas et al.: Lancet 1998, 351:472-477., and Yuen K Y, et al. Lancet 1998, 351:467-471.) the potential for emergence of a pandemic influenza was recognized (de Jong et al. Nature 1997, 389:554.). Mass slaughtering of poultry appeared to halt the outbreak. However, H5N1 viruses of various genotypes have spread across Southeast Asia and have continued to cause disease outbreaks in poultry and aquatic birds. Toward the end of 2003, a single genotype (the “Z-genotype”) became dominant and was responsible for outbreaks in Indonesia, Thailand and Vietnam throughout 2004 (Li et al. Nature 2004, 430:209-213.), causing death in ˜50% of the confirmed cases.

Since this time, the H5N1 influenza epidemic of the Asian bird population has continued. In addition, infection of migratory birds has resulted in increased global spread of the virus with reports of H5N1 influenza causing mortality in poultry and aquatic birds throughout Asia, Europe and Africa. From the beginning, the ability of H5N1 viruses to cross the species barrier was evident not only from the cases of human infection, but also from infection and mortality in domestic cats, captive tigers and leopards (Keawcharoen et al. Emerg Infect Dis 2004, 10:2189-2191. and Kuiken et al Science 2004, 306:241.). Taken together, the increased incidence of human infection, coupled with evidence of expanding host range and widespread distribution of H5N1 viruses has heightened concern that acquisition of the properties necessary, through mutation or genetic reassortment, for human-to-human transmission is only a matter of time. Should this occur, humans would have virtually no immunity to such novel viruses which may result in a human influenza pandemic of potentially catastrophic proportions.

Currently, control of influenza relies on two options, vaccination or antiviral drug treatment with vaccination being the preferred option. However, the high virulence of H5N1 influenza has inhibited the development of vaccines using traditional approaches (Stephenson et al. Lancet Infect Dis 2004, 4:499-509.) as the patients actually die from their own hyper-immune response rather than the virus itself. Hence, if vaccination with an antigen is used there is a high chance the body will go into a hyper-immune response. Other approaches for vaccine production, such as reverse genetics, DNA vaccination and the use of recombinant hemagglutinin (Webby et al. Lancet 2004, 363:1099-1103. and Wood and Robertson Nat Rev Microbiol 2004, 2:842-847.), have been met with varying degrees of success but a human vaccine ready for commercial production is still not available. Therefore, should a pandemic arise due to H5N1, the lack of an effective vaccine means containment would rely solely on the effectiveness of antiviral drugs and physical measures to inhibit viral spread such as social distancing.

For treatment of influenza two classes of antiviral drugs are presently available:

1. M2 ion channel inhibitors (amantadine and rimantadine); and
2. neuraminidase inhibitors (NAIs; oseltamivir and zanamivir).

The presence of H5N1 viruses resistant to the M2 inhibitors (Li et al. Nature 2004, 430:209-213, and Hien et al. N Engl J Med 2004, 350:1179-1188), means their use cannot be relied upon. The neuraminidase inhibitors are currently viewed as the best choice for prophylaxis against—and clinical management of—disease due to H5N1 virus. Its efficacy in both uses is still unclear due to a lack of human data. Recent studies in mice have highlighted prophylactic efficacy against infection by H5N1 virus isolated from Vietnam (Yen et al. J. Infect Dis 2005, 192:665-672.). However, reports have also appeared detailing the development of oseltamivir resistance during treatment of H5N1 infected patients and isolation of drug resistant H5N1 virus in the same region (de Jong et al: N Engl J Med 2005, 353:2667-2672, and Le et al: Nature 2005, 437:1108-1108.). While the current data does not deter stockpiling of oseltamivir for pandemic response, it does suggest that alternative strategies for prophylaxis or treatment are needed.

Hemagglutinin is an antigenic glycoprotein found on the surface of the influenza viruses as well as many other bacteria and viruses. It is responsible for binding the virus to the cell that is being infected. Previous studies examining the antigenic sites of hemagglutinin of influenza A H3N2 have identified two protruding loops, residues 140-146 (140s loop) and 155-164 (150s loop), located near the receptor-binding site that are antibody-binding sites for potent neutralizing antibodies against this virus (Wiley et al. Nature 1981, 289:373-378.). These loops are prone to antigenic drift and mutation introducing a potential glycosylation into this region inhibits antibody binding (Wiley et al. Nature 1981, 289:373-378, and Caton et al. Cell 1982, 31:417-427.). These antigenic loops have also been identified in the hemagglutinin of H5N1 viruses by structural determination (Ha et al. Embo J 2002, 21:865-875.). H5N1 viruses isolated from human cases throughout late 2003 and 2004 were known to differ in the antigenic loop located above the receptor binding site, with a potential glycosylation site in the latter (Hoffmann et al. Proc Natl Acad Sci USA 2005, 102:12915-12920.). Antibodies binding to this antigenic loop are neutralizing due to steric hindrance of the interaction between the receptor binding site of HA and its receptor located on the cell surface (Skehel and Wiley Annu Rev Biochem 2000, 69:531-569.), glycosylation of this loop may inhibit binding of the antibody destroying its virus neutralizing properties. Ideally, a neutralizing antibody whose epitope determinants are not within the antigenic loops and less prone to mutation may somewhat overcome this issue, but the ability to identify such an antibody may be difficult.

A potential drawback to the use of antibodies is the current high cost of large scale antibody production. This raises the costs of treatments utilizing antibodies, such as for RSV infection and autoimmune disease, to several thousands of dollars per treatment.

The present invention seeks to ameliorate the above mentioned problems by providing an antibody specific to hemagglutinin capable of neutralizing influenza viruses and methods of making and using the same.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### SUMMARY

The invention provides an antibody specific to hemagglutinin capable of neutralizing influenza viruses and methods of making and using the same.

The antibody of the invention is further directed to the 140s protruding loop of the hemagglutinin and may be capable of neutralizing influenza virus. The antibody may be monoclonal and or humanized. The antibody may include an expression product of SEQ ID NO. 1, SEQ ID NO. 2, SEQ ID NO. 3 or SEQ ID NO. 4. The antibody may include SEQ ID NO. 5, SEQ ID NO. 6, SEQ ID NO. 7 or SEQ ID NO. 8. The antibody may be expressed from a nucleotide selected from the group of SEQ ID NO. 1, SEQ ID NO. 2, SEQ ID NO. 3 or SEQ ID NO. 4. The antibody may be selected from the group SEQ ID NO. 5, SEQ ID NO. 6, SEQ ID NO. 7 or SEQ ID NO. 8.

The invention is derived from the surprising results of a comparative analysis of the amino acid sequences of numerous H5N1 hemagglutinins identified over the last few years. There is antigenic drift and mutation in the protruding loops of hemagglutinins. Surprisingly, however, the genetic drift in these regions and the introduction of a potential glycosylation site in the 150s loop of virus isolated in Vietnam during 2004 appears to occur more in the 150s loop with less in the 140s loop.

The present invention also provides a method of treating a patient to at least affect an influenza virus infection, which comprises the step of: contacting the infection with (a) an antibody specific to hemagglutinin or (b) an antibody specific to the 140s protruding loop of hemagglutinin. Preferably, the antibody interferes with influenza viral infection by means that neutralize the virus.

An alternative form of the present invention resides in the use of an antibody specific to hemagglutinin or an antibody specific to the 140s protruding loop of hemagglutinin for the treatment of an influenza virus infection, preferably the use at least affects an influenza virus infection.

The present invention also relates to compositions including pharmaceutical compositions comprising a therapeutically effective amount of (a) an antibody specific to hemagglutinin or (b) an antibody specific to the 140s protruding loop of hemagglutinin. As used herein a compound will be therapeutically effective if it is able to affect an influenza virus infection.

The invention also provides a method of diagnosing an influenza viral infection, comprising the steps of the determining an amount of hemagglutinin or the amount of a 140s protruding loop of hemagglutinin in body fluids sampled from a person suspected of having an influenza viral infection.

Accordingly, the methods and compounds described herein may be used in diagnostic, prophylactic and therapeutic methods. Other aspects and advantages of the invention will become apparent to those skilled in the art from a review of the ensuing description, which proceeds with reference to the following illustrative drawings of preferred embodiments.

### BRIEF DESCRIPTION OF THE DRAWINGS

FIG. 1—Humanization of Mouse mAbs VN04-2 and VN04-3

A, Diagrammatic representation of the expression vector used to create chimeric antibodies; CL and CH refer to the constant regions of the human Kappa light and human IgG1, respectively; L refers to the leader sequence. B, ELISA to show presence of human constant regions, antibodies bound to immunosorbent plates were detected using secondary antibodies specific for human IgG and mouse IgG. Following addition of TMB substrate absorbance was measured at 450 nm.

FIG. 2—Prophylactic Efficacy of VN04-2- and VN04-3-huG1 in Mice

Mice were challenged with a lethal dose (10 MLD50) of fully virulent A/Vietnam/1203/04 24 h after the introduction of 1, 5, or 10 mg/kg bodyweight of antibody. The percentage of initial body weight after challenge is indicated for VN04-2-huG1 (A) and VN04-3-huG1 (B) periodically over 15 days. Each data point represents the average of 5 mice. Survival of challenged mice was observed for 21 days after challenge and indicates the level of protection from mortality (C).

FIG. 3—Therapeutic Efficacy of VN04-2-huG1 in Mice

Mice were inoculated with a lethal dose (10 MLD50) of A/Vietnam/1203/04 virus 24 h, followed by the introduction of 1, 5, or 10 mg/kg bodyweight of VN04-2-huG1 antibody one (A and B) and three (C and D) days post infection. The percentage of initial body weight was monitored periodically over 15 days (B and D) and each data point represents the average of 5 mice. Survival of mice was observed for 21 days following infection and indicates the level of protection from mortality (A and C).

FIG. 4. —Sequence of the Antibody Variable Regions Used to Construct VN04-2-HuG1 and VN04-3-HuG1

(A) alignment of the nucleic acid and amino acid of the heavy chain of the humanized monoclonal antibody VN04-2-HuG1 showing the variable regions, (B) alignment of the nucleic acid and amino acid of the light chain of the humanized monoclonal antibody VN04-2-HuG1 showing the variable regions, (C) alignment of the nucleic acid and amino acid of the heavy chain of the humanized monoclonal antibody VN04-3-HuG1 showing the variable regions, (D) alignment of the nucleic acid and amino acid of the light chain of the humanized monoclonal antibody VN04-3-HuG1 showing the variable regions.

FIG. 5. —ELISA Detection of H5 Hemagglutinin Protein from Several Isolates of H5N1 Using the VN04-2 Antibody.

ELISA detection was performed on equal amounts of each hemagglutinin from several H5N1 isolates. These isolates were chosen from all of the publicized hemagglutinin protein sequences of H5N1 viruses isolated in 2005 and 2006 as they are representative of mutations in the 140s loop of the Hemagglutinin protein. ELISA established the ability of VN04-2 to bind the hemagglutinins tested.

### DETAILED DISCLOSURE

The invention is derived from the surprising results of a comparative analysis of the amino acid sequences of numerous H5N1 hemagglutinins identified over the last few years. There is antigenic drift and mutation in the protruding loops of hemagglutinins. Surprisingly, however, the genetic drift in these regions and the introduction of a potential glycosylation site in the 150s loop of virus isolated in Vietnam during 2004 appears to occur more in the 150s loop with less in the 140s loop. The lysine at position 140 remains constant in all the strains examined.

Consistent with the invention there are provided (a) an antibody specific to hemagglutinin and, or (b) an antibody specific to the 140s protruding loop of hemagglutinin. Exemplary antibodies include polyclonal, monoclonal, humanized, bispecific, and hetero conjugate antibodies.

The following embodiments are encompassed by the present invention:
1. An immunoglobulin that specifically binds to the 140s protruding loop of the hemagglutinin.
2. The immunoglobulin of embodiment 1, wherein said immunoglobulin comprises an immunoglobulin heavy chain comprising a variable domain comprising an amino acid sequence selected from the group consisting of SEQ ID NOS: 5 and 7.
3. The immunoglobulin of embodiment 1, wherein said immunoglobulin comprises an immunoglobulin light chain comprising a variable domain comprising an amino acid sequence selected from the group consisting of SEQ ID NOS: 6 and 8.
4. The immunoglobulin of anyone of embodiments 1 through 3, wherein said immunoglobulin is an IgG1 kappa immunoglobulin.
5. The immunoglobulin of embodiment 4, wherein said immunoglobulin comprises a human IgG1 constant region within a heavy chain of said immunoglobulin and a human constant region within a light chain of said immunoglobulin.
6. The immunoglobulin of embodiment 5, wherein said immunoglobulin comprises fully or partially human framework regions within the variable domain of said heavy chain and within the variable domain of said light chain.
7. The immunoglobulin of embodiment 5, wherein said immunoglobulin comprises murine framework regions within the variable domain of said heavy chain and within said light chain.
8. The immunoglobulin of embodiment 1, wherein said hemagglutinin is derived from type A influenza strain H5N1 or Z-genotype of type A influenza strain H5N1.
9. An immunoglobulin that specifically binds to the 140s protruding loop of hemagglutini, wherein said immunoglobulin comprises a variable heavy (VH) domain selected from the group consisting of
   (a) a polypeptide comprising the amino acid sequence of SEQ ID NO: 5 or 7;
   (b) a polypeptide having at least 90% sequence identity to the amino acid sequence of SEQ ID NO: 5 or 7, wherein said immunoglobulin specifically binds to the 140s protruding loop of hemagglutinin;
   (c) a polypeptide that is encoded by the nucleotide sequence of SEQ ID NO: 1 or 3; or
   (d) a polypeptide that is encoded by a nucleotide sequence that is at least 90% identical to the nucleotide sequence of SEQ ID NO: 1 or 3, wherein said immunoglobulin specifically binds to the 140s protruding loop of hemagglutin.
10. The immunoglobulin of embodiment 9, wherein the immunoglobulin comprises an immunoglobulin light chain comprising a variable domain selected from the group consisting of:
   (a) a polypeptide comprising the amino acid sequence of SEQ ID NO: 6 or 8;
   (b) a polypeptide having at least 90% sequence identity to the amino acid sequence of SEQ ID NO: 6 or 8, wherein said immunoglobulin specifically binds to the 140s protruding loop of hemagglutinin;
   (c) a polypeptide that is encoded by the nucleotide sequence of SEQ ID NO: 2 or 4; or
   (d) a polypeptide that is encoded by a nucleotide sequence that is at least 90% identical to the nucleotide sequence of SEQ ID NO: 2 or 4, wherein said immunoglobulin specifically binds to the 140s protruding loop of hemagglutin.
11. The immunoglobulin of any one of embodiments 1 through 10, wherein said immunoglobulin is conjugated to an agent selected from the group consisting of a therapeutic agent, a prodrug, a peptide, a protein, an enzyme, a virus, a lipid, a biological response modifier, a pharmaceutical agent, and PEG.
12. A composition comprising the immunoglobulin of any one of embodiments 1 through 11, and a carrier.
13. An isolated polynucleotide comprising a nucleic acid encoding a variable heavy (VH) domain of an immunoglobulin heavy chain, wherein said VH domain is selected from the group consisting of:
   (a) a nucleic acid molecule comprising the nucleotide sequence of SEQ ID NO: 1 or 3;
   (b) a nucleic acid molecule comprising a nucleotide sequence having at least 90% sequence identity to the nucleotide sequence of SEQ ID NO: 1 or 3, wherein said immunoglobulin specifically binds to the 140s protruding loop of hemagglutinin;
   (c) a nucleic acid that encodes a polypeptide comprising the amino acid sequence of SEQ ID NO: 5 or 7; or
   (d) a nucleic acid molecule that encodes a polypeptide that is at least 90% identical to the amino acid sequence of SEQ ID NO: 5 or 7, wherein said immunoglobulin specifically binds to the 140s protruding loop of hemagglutinin.
14. An isolated polynucleotide comprising a nucleic acid encoding a variable light (VL) domain of an immunoglobulin light chain, wherein said VL domain is selected from the group consisting of:
   (a) a nucleic acid molecule comprising the nucleotide sequence of SEQ ID NO: 2 or 4;
   (b) a nucleic acid molecule comprising a nucleotide sequence having at least 90% sequence identity to the nucleotide sequence of SEQ ID NO: 2 or 4, wherein said immunoglobulin specifically binds to the 140s protruding loop of hemagglutinin;
   (c) a nucleic acid that encodes a polypeptide comprising the amino acid sequence of SEQ ID NO: 6 or 8; or
   (d) a nucleic acid molecule that encodes a polypeptide that is at least 90% identical to the amino acid sequence of SEQ ID NO: 6 or 8, wherein said immunoglobulin specifically binds to the 140s protruding loop of hemagglutinin.
15. A method for preventing or treating influenza virus infection in a subject comprising administering to said subject an effective amount of a composition comprising the immunoglobulin according to any one of embodiments 1 through 10.
16. The method of embodiment 15, wherein said influenza virus comprises type A influenza strain H5N1 or the Z-genotype of type A influenza strain H5N1.
17. A pharmaceutical composition comprising the immunoglobulin according to any one of embodiments 1 through 12.
18. A method of diagnosing infection with an influenza virus in a subject comprising contacting the subject with a composition according to any one of embodiments 1 through 11 and detecting the presence of hemagglutinin.

### Polyclonal Antibodies

The antibodies of the invention may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant.

Typically, the immunizing agent and/or adjuvant will be injected in the mammal by multiple subcutaneous or intraperitoneal injections. The intensity of the response is determined by several factors including the size of the immunogen molecule, its chemical characteristics, and how different it is from the animal's own proteins. Most natural immunogens are proteins with a molecular weight above 5 kDa that come from sources phylogenically far removed from the host animal (i.e., human proteins injected into rabbits or goats). It is desirable to use highly purified proteins as immunogens, since the animal will produce antibodies to even small amounts of impurities present as well as to the major component. The antibody response increases with repeated exposure to the immunogen, so a series of injections at regular intervals is needed to achieve both high levels of antibody production and antibodies of high affinity.

To the extent that the antagonist is an antibody that engage the 140s loop of the hemagglutinin in an influenza virus preventing viral infection the immunogen will be an selected from amino acids comprising the 140s loop domain from hemagglutinin Preferably, the amino acid sequence will be selected from the region of about 94 to 156 in the hemagglutinin protein. Sequences of at least 5, 6, 7, 8, 9, 10, 15, 20, 25, 30 amino acids from this region will generally be used to generate those antibodies. Desirably, the sequence selected will generate an antibody that specifically interferes with binding of hemagglutinin to the host cell receptor.

Not all immunogenic molecules will however generate the level of antibody desired. To increase the intensity of the immune response immunogens are combined with complex mixtures called adjuvants. Adjuvants are a mixture of natural or synthetic compounds that, when administered with antigens, enhance the immune response. Adjuvants are used to (1) stimulate an immune response to an antigen that is not inherently immunogenic, (2) increase the intensity of the immune response, (3) preferentially stimulate either a cellular or a humoral response (i.e., protection from disease versus antibody production). Examples of adjuvants which may be employed include Freund's complete adjuvant and MPL-TDM adjuvant (monophosphoryl Lipid A, synthetic trehalose dicorynomycolate). A more extensive discussion of adjuvants and their use in immunization protocols is given in Immunology Methods Manual, vol. 2, I. Lefkovits, ed., Academic Press, San Diego, Calif., 1997, ch. 13. Immunology Methods Manual is available as a four volume set, (Product Code Z37,435-0); on CD-ROM, (Product Code Z37,436-9); or both, (Product Code Z37,437-7)

If the immunogen is still unable to generate an acceptable response, it may be conjugated to a carrier protein that is more immunogenic. Small molecules such as drugs, organic compounds, and peptides and oligosaccharides with a molecular weight of less than 2-5 kDa like, for example, small segments if hemagglutinin in their core structure, are not usually immunogenic, even when administered in the presence of adjuvant. In order to generate an immune response to these compounds, it is necessary to attach them to a protein or other compound, termed a carrier that is immunogenic. When attached to a carrier protein the small molecule immunogen is called a hapten. Haptens are also conjugated to carrier proteins for use in immunoassays. The carrier protein provides a means of attaching the hapten to a solid support such as a microtiter plate or nitrocellulose membrane. When attached to agarose they may be used for purification of the anti-hapten antibodies. They may also be used to create a multivalent antigen that will be able to form large antigen-antibody complexes. When choosing carrier proteins, remember that the animal will form antibodies to the carrier protein as well as to the attached hapten. It is therefore relevant to select a carrier protein for immunization that is unrelated to proteins that may be found in the assay sample. If haptens are being conjugated for both immunization and assay, the two carrier proteins should be as different as possible. This allows the antiserum to be used without having to isolate the anti-hapten antibodies from the anti-carrier antibodies.

Where the immunizing agent is hemagglutinin segment such as from the 140s loop preferably the hemagglutinin segment is conjugated to a protein known to be immunogenic in the mammal being immunized.

Examples of such immunogenic proteins include but are not limited to keyhole limpet hemocyanin (KLH), serum albumin, bovine thyroglobulin, soybean trypsin inhibitor, and a toxoid, for example tetanus toxoid.

KLH is a respiratory protein found in molluscs. Its large size makes it very immunogenic, and the large number of lysine residues available for conjugation make it very useful as a carrier for haptens. The phylogenic separation between mammals and molluscs increases the immunogenicity and reduces the risk of cross-reactivity between antibodies against the KLH carrier and naturally occurring proteins in mammalian samples.

KLH is offered both in its native form, for conjugation via amines, and succinylated, for conjugation via carboxyl groups. Succinylated KLH may be conjugated to a hapten containing amine groups (such as a peptide) via cross-linking with carbodiimide between the newly introduced carboxyl groups of KLH and the amine groups of the hapten.

Protocols for conjugation of haptens to carrier proteins may be found in Antibodies: A Laboratory Manual, E. Harlow and D. Lane, ed., Cold Spring Harbor Laboratory (Cold Spring Harbor, N.Y., 1988) pp. 78-87 (Product Code A 2926)

The immunization protocol may be selected by one skilled in the art without undue experimentation. Protocols for preparing immunogens, immunization of animals, and collection of antiserum may be found in Antibodies: A Laboratory Manual, E. Harlow and D. Lane, ed., Cold Spring Harbor Laboratory (Cold Spring Harbor, N.Y., 1988) pp. 55-120 (Product Code A 2926).

### Monoclonal Antibodies

The antibodies may, alternatively, be monoclonal antibodies. Monoclonal antibodies may be prepared using hybridoma methods, such as those described by Kohler and Milstein (1975), Nature 256:495. In a hybridoma method, a mouse, hamster, or other appropriate host animal, is typically immunized with an immunizing agent as described above to elicit lymphocytes that produce or are capable of producing antibodies that will specifically bind to the immunizing agent. Alternatively, the lymphocytes may be immunized in vitro.

Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. (1984) Immunol., 133:3001).

The culture medium in which the hybridoma cells are cultured can then be assayed for the presence of monoclonal antibodies directed against hemagglutinin and/or the 140s loop of hemagglutinin or any of the sequences SEQ ID No. 5 to 8.

After the desired hybridoma cells are identified, the clones may be subcloned by limiting dilution procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively, the relevant cysteine residues are substituted with another amino acid residue or are deleted so as to prevent cross-linking. The light chain may be further selected from SEQ ID No. 2, 6, 4 or 8. The heavy chain may be further selected from SEQ ID No. 1, 3, 5 or 7.

In vitro methods are also suitable for preparing monovalent antibodies. Digestion of antibodies to produce fragments thereof, particularly, Fab fragments, can be accomplished using routine techniques known in the art.

### Human and Humanized Antibodies

The antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding sub-sequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin.

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., (1986) Nature, 321:522-525; Riechmann et al., (1988) Nature, 332:323-327; Verhoeyen et al., (1988) Science 239:1534-1536], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. (1991) Mol. Biol. 227:381; Marks et al., (1991) J. Mol. Biol., 222:581]. The techniques of Cole et al. and Boerner et al. are also available for the preparation of human monoclonal antibodies (Cole et al., (1985) Monoclonal Antibodies and Cancer Therapy, Alan R. Liss, p. 77 and Boerner et al., (1991) J. Immunol., 147(1):86-95]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., (1992) Bio/Technology 10, 779-783; Lonberg et al., (1994) Nature 368 856-859; Morrison, (1994) Nature 368, 812-13; Fishwild et al., (1996) Nature Biotechnology 14, 845-51; Neuberger, (1996) Nature Biotechnology 14, 826; Lonberg and Huszar, (1995) Intern. Rev. Immunol. 13 65-93.

### Bispecific Antibodies

Bispecific antibodies are monoclonal, preferably human or humanized, antibodies that have binding specificities for at least two different antigens. In the present case, one of the binding specificities is for hemagglutinin and/or a segment of hemagglutinin comprising portions of the 140s loop, the other one is for another compound having hemagglutinin.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, (1983) Nature, 305:537-539]. The light chain may be further selected from SEQ ID No. 2, 6, 4 or 8. The heavy chain may be further selected from SEQ ID No. 1, 3, 5 or 7.

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Immunoconjugates

The invention also pertains to immunoconjugates comprising an antibody conjugated to a cytotoxic agent such as a chemotherapeutic agent, toxin (e.g., an enzymatically active toxin against hemagglutinin), or a radioactive isotope (i.e., a radioconjugate).

Conjugates of the antibody and cytotoxic agent are made using a variety of bifunctional protein-coupling agents such as N-succinnimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

### Method of Treatment and or Use of the Antibodies of the Invention

The present invention also provides a method of treating a patient to at least affect an influenza virus infection, which comprises the step of contacting the infection with (a) an antibody specific to hemagglutinin or (b) an antibody specific to the 140s protruding loop of hemagglutinin. Preferably, the antagonist interferes with viral infection by means that neutralize the virus.

An alternative form of the present invention resides in the use of an antibody specific to hemagglutinin or an antibody specific to the 140s protruding loop of hemagglutinin for the treatment of an influenza virus infection, preferably the use at least affects a virus infection.

An influenza virus infection or viral infection may include, all types of known and new influenza viruses that contain a hemagglutinin protein such as influenza A, which may include influenza A H5N1 or Z-genotype of influenza A H5N1.

“Treatment” and “treat” and synonyms thereof refer to both therapeutic treatment and prophylactic or preventative measures, wherein the object is to prevent or slow down (lessen) an influenza virus infection, in particular an influenza A virus infection. Treatment may include prophylactic passive immunization or immunotherapy treatment of a patent. Those in need of such treatment include those already with an influenza infection as well as those prone to getting it or those in whom an influenza virus infection is to be prevented. Immunization may be introduced in healthy individuals particularly those at risk of contracting an influenza viral infection either in an area where several cases of the viral infection have occurred or a person who has recently been to an area where several cases of the viral infection have occurred. Immunization may also be introduced to those more susceptible to infection such as the elderly or children.

In one example of the invention passive immunization is used. Passive immunization involves treating an animal infected with a microorganism by administering the animal with an antibody to a protein of the microorganism. This is quite different from the traditional approach to immunization where an antigen either alone or with an adjuvant is given to an animal to elicit an immune response from the host where the host animal produces its own antibodies in response to the vaccination. In passive immunization the host animal is given the ready made antibody. Where the host animal is human a humanized antibody will be more readily accepted by the host. Passive immunization is particularly effective where the antigen is highly pathogenic and capable of a hyper-immune response in the host animal as there is a chance traditional vaccination with the antigen my cause a hyper-immune response in the host animal that could mimic the symptoms of the disease and or even result in death of the host. A host animal may be any animal such as but not limited to aves (birds), mammals, humans etc.

Given the specificity and efficacy of the antibodies used in the examples, it would not be unwarranted to suggest that passive antibody therapy with these antibodies could be used in a similar manner to antiviral drugs, drastically reducing the amount of antibody needed for stockpiling to an achievable level that may be quite cost competitive.

As used herein a “therapeutically effective amount” of a compound will be an amount of active agent that is capable of preventing or at least slowing down (lessening) an influenza infection, in particular an influenza A virus infection. Dosages and administration of an antagonist of the invention in a pharmaceutical composition may be determined by one of ordinary skill in the art of clinical pharmacology or pharmacokinetics. See, for example, Mordenti and Rescigno, (1992) Pharmaceutical Research, 9:17-25; Morenti et al., (1991) Pharmaceutical Research, 8:1351-1359; and Mordenti and Chappell, “The use of interspecies scaling in toxicokinetics” in Toxicokinetics and New Drug Development, Yacobi et al. (eds) (Pergamon Press: NY, 1989), pp. 42-96. An effective amount of the antibody to be employed therapeutically will depend, for example, upon the therapeutic objectives, the route of administration, and the condition of the mammal. Accordingly, it will be necessary for the therapist to titer the dosage and modify the route of administration as required to obtain the optimal therapeutic effect. A typical daily dosage might range from about 10 ng/kg to up to 100 mg/kg of the mammal's body weight or more per day, preferably about 1 μg/kg/day to 10 mg/kg/day. Doses may include an antibody amount any where in the range of 0.1 to 20 mg/kg of bodyweight or more preferably 1, 5, 10 mg/kg of bodyweight.

### Compositions of the Invention

Antibodies produced according to the invention, can be administered for the treatment of viral infection in the form of pharmaceutical compositions.

Thus, the present invention also relates to compositions including pharmaceutical compositions comprising a therapeutically effective amount of (a) an antibody specific to hemagglutinin and, or (b) an antibody specific to the 140s protruding loop of hemagglutinin. As used herein a compound will be therapeutically effective if it is able to affect viral infection.

Pharmaceutical forms of the invention suitable for injectable use include sterile aqueous solutions such as sterile phosphate-buffered saline (where water soluble) or dispersions and sterile powders for the extemporaneous preparation of sterile injectable solutions and or one or more carrier. Alternatively, injectable solutions may be delivered encapsulated in liposomes to assist their transport across cell membrane. Alternatively or in addition such preparations may contain constituents of self-assembling pore structures to facilitate transport across the cellular membrane. It must be stable under the conditions of manufacture and storage and must be preserved against the contaminating/destructive action of microorganisms such as, for example, bacteria and fungi.

The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as, for example, lecithin, by the maintenance of the required particle size in the case of dispersion and by the use of surfactants. Preventing the action of microorganisms in the compositions of the invention is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and freeze-drying, to yield a powder of the active ingredient plus any additional desired ingredient from previously sterile-filtered solution thereof.

The active ingredient may be held within a matrix which controls the release of the active agent. Preferably, the matrix comprises a substance selected from the group consisting of lipid, polyvinyl alcohol, polyvinyl acetate, polycaprolactone, poly(glycolic)acid, poly(lactic)acid, polycaprolactone, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and combinations thereof and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety. Preferably, the matrix sustainedly releases the antibody.

Pharmaceutically acceptable carriers and/or diluents may also include any and all solvents, dispersion media, coatings, antibacterials and/or antifungals, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated.

### Neutralizing Humanized Monoclonal Antibodies Specific for the Hemagglutinin of a Z-Genotype Influenza A H5N1 Virus

Passive immunization may provide an alternative strategy for both prophylaxis and treatment against pandemic influenza, however use of animal derived antibodies can result in severe anaphylactoid side effects and the induction of human anti-species specific antibody responses which limits the efficacy of the antibodies with repeated use. We report here, the protective efficacy of neutralizing humanized monoclonal antibodies specific for the hemagglutinin of a Z-genotype influenza A H5N1 virus in mice when used as prophylaxis before—, and treatment following—lethal challenge with fully pathogenic H5N1 virus.

### MAbs to Hemagglutinin of A/Vietnam/1203/04 (H5N1)

MAbs to the HA of A/Vietnam/1203/04 and A/Hong Kong/213/03 were prepared in mice immunized with attenuated versions of the respective H5N1 virus generate by reverse genetics using a modification of the method described by Kohler and Milstein (1975), Nature, 256:495.

### Construction of Human IgG1 Constant Region Expression Vector

Design of the expression vector for human IgG1 was based on that described by Jostock et al, 2004 (J Immunol Methods, 289:65-80.). Briefly, a synthetic construct containing the recognition sites for ApaL1, Pst1, Asc1, Nco1, Mfe1, Xho1 and Xba1 with a synthetic secretion leader sequence between Nco1 and Mfe1 was used to replace the multiple cloning site, myc-tag and ER retention signal of pCMV/myc/ER (Invitrogen). The internal ribosome entry site (IRES) of encephalomyocarditis virus was inserted between Asc1 and Nco1 following amplification from pIRES (Clontech) to introduce the relevant sites. For insertion of the human antibody constant regions, cDNA clones (I.M.A.G.E. Consortium cDNA clones Lennon et al. Genomics 1996, 33:151-152.) encoding the Kappa light chain (Clone ID 6279986) and the IgG1 heavy chain (Clone ID 6281248) were amplified to allow insertion of the constant regions between Pst1 and Asc1; and Xho1 and Xba1, respectively. Recognition sites within the antibody constant regions affecting cloning were removed by site-directed mutagenesis.

### Cloning of Chimeric IgG1 Expression Vectors

To isolate the cDNA encoding the variable regions of the monoclonal antibodies, mRNA was prepared from hybridoma cells and used in first strand cDNA synthesis with random hexa-nucleotides. The total cDNA was then used as template in reactions to amplify both the variable heavy and light chain using the primers and protocols of the mouse scFv recombinant antibody phage system (Amersham Biosciences) with the resulting products cloned into pCR-Script (Stratagene) for sequencing. Variable region specific primers were used to amplify both the heavy and light chain variable regions with addition of recognition sites to allow cloning between the Mfe1 and Xho1; and ApaL1 and Pst1 sites of the human IgG1 constant region expression vector, respectively. Cloning according to this protocol produces constructs from which expression gives rise to chimeric antibodies containing the mouse variable and human constant regions.

### Transient Expression of Chimeric Antibodies and Purification

Chimeric antibodies were expressed using the FreeStyle™ 293 expression system (Invitrogen) to obtain antibodies produced in a defined, serum-free medium. Constructs encoding chimeric IgG1 were transfected into 293-F cells by use of 293fectin (Invitrogen). Supernatants were collected 120 h after transfection and proteins purified using protein A sepharose beads (Amersham). Purity of IgG was confirmed using SDS-PAGE analyses. ELISA using HRP labeled anti-mouse IgG (Sigma) and anti-human IgG (Accurate Chemical & Scientific Corporation) was used to highlight the introduction of human IgG constant regions

### Virus Neutralization and HI Tests

Virus neutralization tests were performed in Madin Darby canine kidney (MDCK) cells and hemagglutinin inhibition (HI) assays were performed with 0.5% chicken red blood cells as previously described (Hoffmann et al. Proc Natl Acad Sci USA 2005, 102:12915-12920). In each HI assay four hemagglutinin units (HAUs) of virus were used and 100 50% tissue culture infective doses (TCID50) were used in each of the virus neutralization tests.

Characterization of H5 Neutralizing mAbs

Monoclonal antibodies against H5N1 viruses of the Z genotype which had shown high titers in HI tests against their respective immunogens were tested for their virus neutralizing capabilities. H5N1 viruses isolated from human cases throughout late 2003 and 2004 were known to differ in the antigenic loop located above the receptor binding site, with a potential glycosylation site in the latter (Hoffmann et al. Proc Natl Acad Sci USA 2005, 102:12915-12920). Antibodies binding to this antigenic loop are neutralizing due to steric hindrance of the interaction between the receptor binding site of HA and its receptor located on the cell surface (Skehel and Wiley Annu Rev Biochem 2000, 69:531-569), glycosylation of this loop may inhibit binding of the antibody destroying it virus neutralizing properties. Therefore virus neutralization was performed with A/Hong Kong/213/03 in addition to A/Vietnam/1203/04 to allow identification of neutralizing mAbs that were not dependant on this region for activity. As highlighted in table 1 both VN04-2 and VN04-3 exhibited similar virus neutralization titers with both of the H5N1 isolates, while VN04-6 and HK03-3 did not. Therefore VN04-2 and VN04-3 were selected for humanization and efficacy studies in a mouse model.

TABLE 1
Virus-neutralization titers of mAbs against HA of H5N1
Virus neutralization assays were performed in MDCK cells.
Titers are the reciprocal lowest dilutions of mAbs that
completely inhibited 100 TCID50
of virus.

mAb to H5N1 HA

| Virus | VN04-2 | VN04-3 | VN04-6 | HK03-3 |
|---|---|---|---|---|
| A/Vietnam/1203/04 | 512 | >512 | >512 | 256 |
| A/HK/213/03 | 512 | >512 | 12 | >512 |

Humanization of H5N1 Neutralizing mAbs

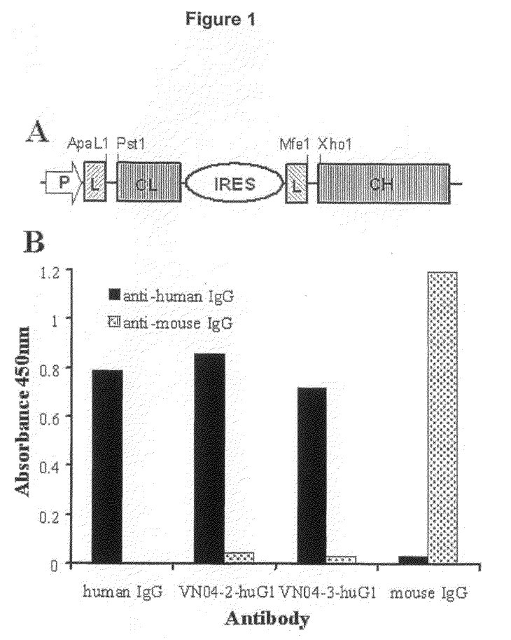

To humanize the mAbs, we constructed chimeric antibodies where the coding regions of the mouse antibody variable domains were fused to the coding region of the constant domains of the human kappa light chain and IgG1 heavy chain using the construct described in FIG. 1A. Both chimeric antibodies (VN04-2-huG1 and VN04-3-huG1) were purified and their purity was confirmed by SDS-PAGE analysis, where resolution characteristics of the antibodies were observed and the preparation determined to be essentially free of contaminants (data not shown). Positive ELISA only in the presence of antibodies specific for human IgG confirmed the humanization of the mouse mAbs (FIG. 1B).

In order to ensure that humanization of the mAbs did not destroy specificity; we examined the performance of the humanized version of the antibodies in HI assays (table 2). For both VN04-2-huG1 and VN04-3-huG1, the titers for the assays against A/Vietnam/1203/04 and A/Hong Kong/213/03 indicated that the specificity was retained.

TABLE 2
HI assay testing of humanized mAb against HA of H5N1
HI assays were performed in microtiter plates with 0.5%
chicken RBC. Titers are the reciprocal lowest dilutions of
antibodies that inhibited hemagglutination caused by 4 HAU
of virus

| | mAb to A/Vietnam/1203/04 HA | | | |
|---|---|---|---|---|
| Virus | VN04-2 | VN04-2-huG1 | VN04-3 | VN04-3-huG1 |
| A/Vietnam/1203/04 | 6400 | 400 | 3200 | 800 |
| A/HK/213/03 | 6400 | 3200 | 6400 | 3200 |

### Epitope Mapping

Mapping of the epitope recognized by VN04-2 and VN04-3 was performed using HI assay data as an indication of antibody recognition, where high titer indicated strong binding and low titers indicates negligible or non existent binding. HI assay data from a recent paper that examined the antigenic properties of multiple H5N1 sublineages, using a number of mAbs against various H5N1 isolates including VN04-2 (referred to as 15A3 in Chen et al. Proc Natl Acad Sci USA 2006, 103:2845-2850), was also included. Alignment of the H5 amino acid sequences was and scattered mutations in positions not common to all of the HI assay negative isolates were not included in the examination.

### Epitope Mapping

To determine the epitope on the H5 hemagglutinin protein recognized by VN04-2 and VN04-3, we examined the amino acid differences between HAs of H5N1 viruses (SEQ ID NOS: 9, 10, 12, 13 and 18), using HI assay results as a measure of antibody binding (table 3). The majority of mutations occurred within the 140s and 150s antigenic loops identified from studies of H3N2 which are positioned direct below and directly above the receptor binding site, respectively [27]. Two positions were identified which differed in all of the HI assay negative isolates, amino acid 94 and amino acid 140 within the 140s loop. However the amino acid at position 94 faces the inside of the HA trimer and is unlikely to affect antibody binding. Therefore the epitope recognized by VN04-2 is most likely within or in close proximity to 140s antigenic loop, with residue 138, 140 and 141 showing significant contribution to the specificity as even viruses with mutation only in this residue of the 140 loop were not inhibited by the antibody in HI assays. Residues of the 140 loop of the hemagglutinin protein that appear to be important for antibody binding specificity are amino acid position 138 as a Glutamine (Q), or amino acid position 140 as a Lysine (K), or amino acid position 141 as a Serine (S), mutations at all 3 amino acid positions lowered the inhibitory effect of VN04-2.

TABLE 3
Epitope mapping of VN04-2
Viruses where HI assay titer data was taken from Chen et al, 2006 [26]
are highlighted by asterix. Residues matching that in A/Vietnam/1203/04
are represented by full stop. Residue numbering refers to the position in
A/Vietnam/1203/04.

| | | Amino acid position in HA1 | | | | | | | |
|---|---|---|---|---|---|---|---|---|---|
| | HI | | | 140s Loop | | | 150s Loop | | |
| Virus | Titer | 94 | 124 | 138 | 140 | 141 | 154 | 155 | 156 |
| A/Vietnam/1203/04 | +++ | D | S | Q | K | S | N | S | T |
| A/Hong Kong/213/03 | +++ | . | . | . | . | . | . | N | A |
| A/Dk/Hong Kong/2986.1/00* | +++ | . | N | . | . | . | . | . | A |
| A/Mdk/Jiang Xi/1653/05* | ++ | . | N | L | . | P | . | . | A |
| A/Mdk/Jiang Xi/2136/05* | − | N | D | . | R | . | . | N | A |
| A/Dk/Vietnam/568/05* | − | N | D | . | N | . | . | D | A |
| A/Qing Hai/05* | − | N | D | . | R | . | . | N | A |

Protection of Mice with Chimeric mAbs

All mouse studies were conducted under applicable laws and guidelines of and after approval from the St. Jude Children's Research Hospital Animal Care and Use Committee. Female 6-8 weeks old C57BL/6 mice (Jackson Laboratories) were housed 5 per cage in ABSL3+ containment. Food and water were provided ad libitum. Mice (5 per group) received the indicated amount of antibody eg 1, 5 and 10 mg/kg of bodyweight in approximately 300 μL of sterile phosphate-buffered saline (PBS) by intraperitoneal (IP) injection. The control group received 300 μL of PBS by IP. For lethal virus challenge, mice were inoculated intranasally with 10 MLD50 (50% mouse lethal dose) in 30 μL of PBS of a fully virulent genetic clone of A/Vietnam/1203/04 virus derived by reverse genetics. This virus is highly pathogenic in mice without prior adaptation and symptoms preceding death are weight loss >30%, general inactivity and the development of hind leg paralysis. For the prophylaxis study, mice received the antibodies at the indicated doses 24 hours prior to lethal virus challenge. To determine therapeutic potential, mice were given a lethal virus dose, followed by the indicated amounts of antibody either one or three days post challenge. Morbidity and mortality were monitored for 21 days and the mice were weighed on days 4, 7, 10, 13 and 15 following virus challenge.

Prophylactic Efficacy of VN04-2- and VN04-3 huG1 Against A/Vietnam/1203/04 Virus In Vivo

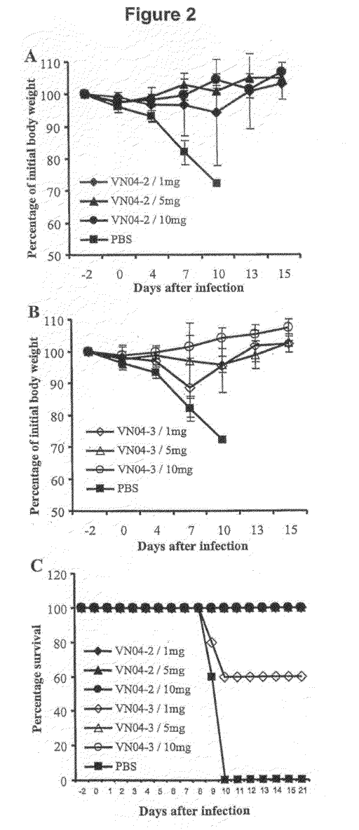

To evaluate prophylactic efficacy of the chimeric antibodies, VN04-2-huG1 and VN04-3-huG1 were introduced into mice at the indicated doses twenty four hours prior to lethal virus challenge. Mice receiving low doses of VN04-2-huG1 antibody (1 mg/kg bodyweight) demonstrated few clinical disease signs including weight loss and death after virus challenge. Only one mouse lost more than 10% of its original bodyweight with full recovery by day 15 (FIG. 2A). Increased amounts of this antibody (5 or 10 mg/kg bodyweight) completely protected mice from disease upon challenge (FIGS. 2, A and C). Prophylactic efficacy was also observed for VN04-3-huG1, although not at the extent of VN04-2-huG1, as three mice receiving 1 mg/kg bodyweight showed significant weight loss of more then 10% (FIG. 2B), two of which were found dead by day 10 after virus challenge (FIG. 2C). Treatment with 5 mg/kg bodyweight of VN04-3-huG1 exhibited similar efficacy as with 1 mg/kg bodyweight of VN04-2-huG1. Finally, 10 mg/kg bodyweight of antibody VN04-3-huG1 completely protected mice from any clinical signs including death after challenge with H5N1 virus.

Therapeutic Efficacy of VN04-2-huG1 Against A/Vietnam/1203/04 Virus In Vivo

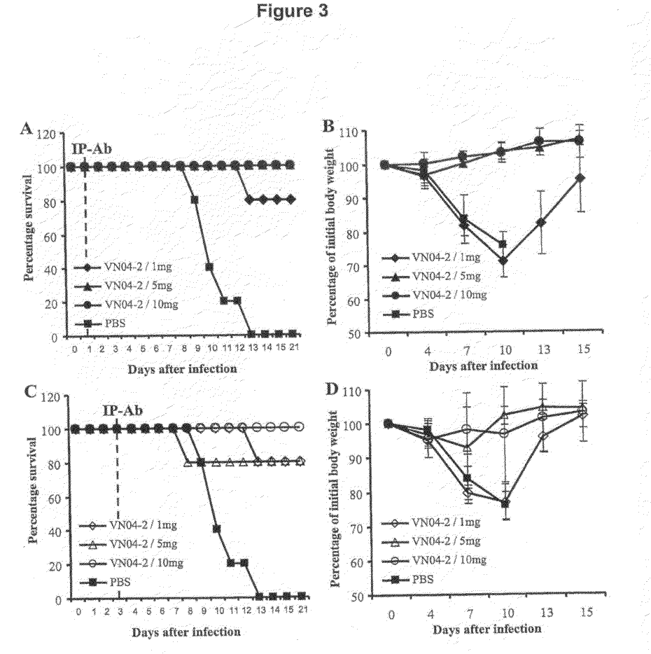

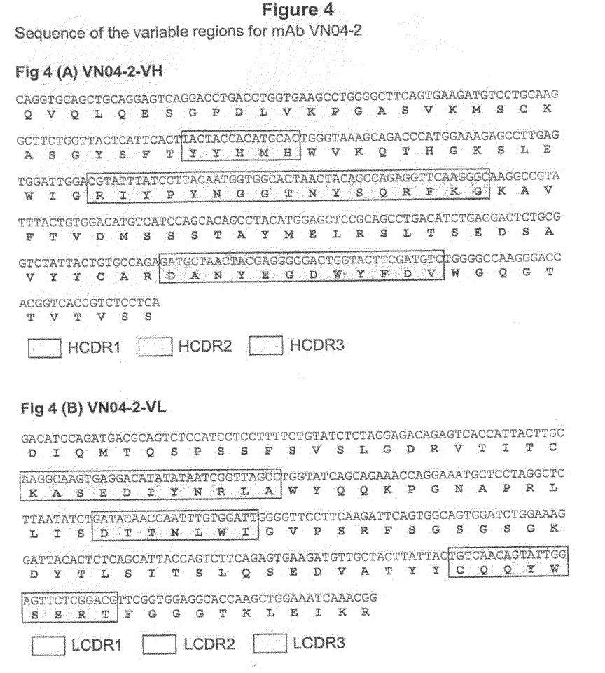

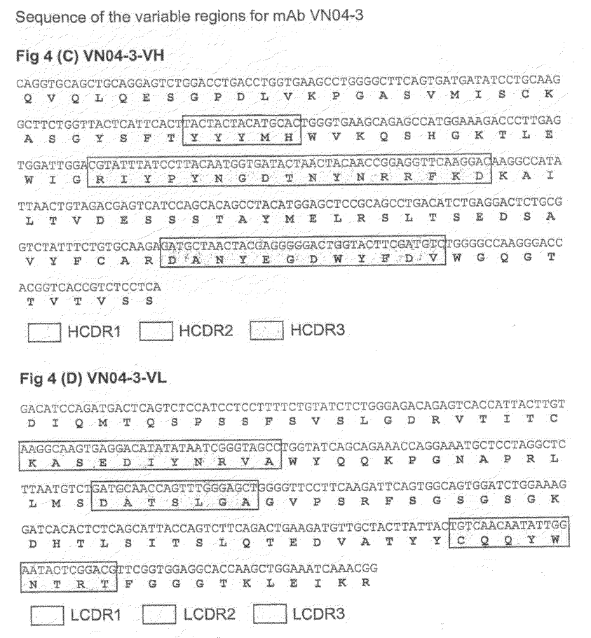

Since VN04-2-huG1 showed greater prophylactic efficacy than VN04-3-huG1, therapeutic efficacy was determined for this antibody alone. The indicated dosages of antibody were introduced one and three days post lethal virus infection (FIG. 3). When the antibodies were given one day after infection (FIGS. 3, A and B), 1 mg/kg bodyweight of VN04-2-huG1 showed 80% protection, the remaining mice did show significant signs of disease but recovered by day 15. The higher doses of antibody (5 or 10 mg/kg bodyweight) completely protected the mice and showed little sign of disease. When antibodies were introduced three days after infection (FIGS. 3, C and D), 10 mg/kg bodyweight of VN04-2-huG1 was required to confer complete protection, with lower doses (1 and 5 mg/kg bodyweight) showing 80% protection. The lower dosages of antibody also showed increased signs of disease; however all of the mice that did not succumb to infection recovered the initial weight loss by day 15.

Humanized monoclonal antibodies (mAbs) that neutralize H5N1 virus could be used as prophylaxis and treatment to aid in the containment of such a pandemic.

Neutralizing mAbs against H5 hemagglutinin were humanized and introduced into C57BL/6 mice (1, 5, or 10 mg/kg bodyweight) one day prior to-, one day post- and three days post-lethal challenge with H5N1 A/Vietnam/1203/04 virus. Efficacy was determined by observation of weight loss as well as survival.

Two mAbs neutralizing for antigenically variant H5N1 viruses, A/Vietnam/1203/04 and A/Hong Kong/213/03 were identified and humanized without loss of specificity. Both antibodies exhibited prophylactic efficacy in mice, however, VN04-2-huG1 performed better requiring only 1 mg/kg bodyweight for complete protection. When used to treat infection VN04-2-huG1 was also completely protective, even when introduced three days post infection, although higher dose of antibody was required.

Prophylaxis and treatment using neutralizing humanized mAbs is efficacious against lethal challenge with A/Vietnam/1203/04, providing proof of principle for the use of passive antibody therapy as a containment option in the event of pandemic influenza.

### Range of Specificity of the VN04-2 Antibody on H5 of Isolates.

The above results demonstrate the ability of VN04-2 and VN04-3 to bind to hemagglutinin from A/Vietnam/1203/04 and A/Hongkong/213/03. Both VN04-2 and VN04-3 provide effective prophylaxis against the A/Vietnam/1203/04 isolate. We demonstrated the ability of VN04-2 to treat established infection with A/Vietnam/1203/04H5Na virus and found efficacy when the antibodies were added up to 3 days post infection.

Based on the data used to identify the mouse monoclonal antibodies which we decided to humanize it was apparent that the 140s loop antigenic region of hemagglutinin was that responsible for binding of both VN04-2 and VN04-3. Alignment of all of the publicized hemagglutinin protein sequences of H5N1 viruses isolated in 2005 and 2006 had shown that this region was indeed prone to mutation. We selected the following H5N1 virus hemagglutinins that had the greatest amount of androgenic drift to determine if the antibody would still be effective in isolates with mutations in the 140s protruding loop. We produced constructs of isolates:

SEQ ID NO. 9—A/Vietnam/1203/04

SEQ ID NO. 11—A/Chicken/Shantou/810/05

SEQ ID NO. 12—A/Qing Hai/05

SEQ ID NO. 14—A/Duck/Vietnam/376/05

SEQ ID NO. 15—A/Chicken/Ivory Coast/1787/06

SEQ ID NO. 16—A/Zhe Jiang/16/06

SEQ ID NO. 17—A/Indonesia/CDC597/06

SEQ ID NO. 19—A/Duck/Guangzhou/20/05

SEQ ID NO. 20—A/Chicken/Indonesia/R60/05

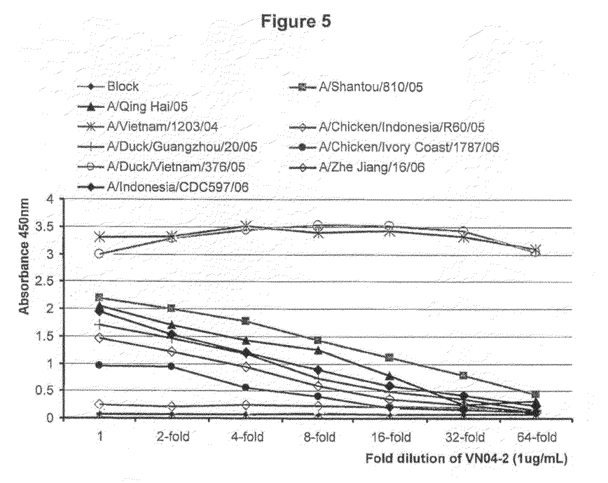

We then expressed the protein in insect cells using recombinant baculovirus, an established method for hemagglutinin protein production in a functional manner. ELISA was performed on equal amounts of each hemagglutinin. It was established that VN04-2 was able to bind all the hemagglutinins tested (FIG. 5). Tabulation of the results together with the protein sequence at the 140s antigenic loop identified the requirements for ‘escape’ from VN04-2 binding (Table 4). Generally, 3 mutations within this region are required to weaken binding of VN04-2, specifically a combination of mutations at position 138, 140 and 141 relative to the 1^{st} amino acid of the mature protein. The results suggest that VN04-2 should bind to H5N1 from all of the known isolates.

TABLE 4
Comparison of ELISA data to 140 antigenic loop protein sequence.
Comparison of HI data and ELISA data in relation to the sequence of the 140s loop. The
sequences are compared to A/Vietnam/1203/04 where a Period (.) indicates the same amino
acid in that location. nd denotes were the assay was not determined

| | Amino Acid position in HA1 of 140s loop | | | | | | | | | |
|---|---|---|---|---|---|---|---|---|---|---|
| H5N1 Influenza A Virus | | HI | | | | | | | | |
| isolate | Clade | titer | ELISA | 136 | 137 | 138 | 139 | 140 | 141 | 142 |
| A/Vietnam/1203/04 | 1 | +++ | +++ | P | Y | Q | G | K | S | S |
| A/HongKong/213/03 | 1 | +++ | nd | . | . | . | . | . | . | . |
| A/CK/Shantou/810/05 | 2.2 | nd | ++ | . | . | . | . | S | . | . |
| A/QingHai/05 | 2.2 | − | ++ | . | . | . | . | R | . | . |
| A/DK/Vietnam/568/05 | 1 | − | nd | . | . | . | . | N | . | . |
| A/DK/Vietnam/376/05 | 1 | nd | +++ | . | . | . | . | N | P | . |
| A/CK/IvoryCoast/1787/ | 2.2 | nd | + | . | H | . | . | R | . | . |
| 06 | | | | | | | | | | |
| A/ZheJiang/16/06 | 2.3 | nd | ++ | . | . | L | . | T | P | . |
| A/Indonesia/CDC597/06 | 2.1 | nd | ++ | . | . | L | . | R | . | . |
| A/MDK/JiangXi/1653/05 | 2.2 | ++ | nd | . | . | L | . | R | . | . |
| A/DK/Guangzhou/20/05 | 2.3 | nd | ++ | . | . | L | . | . | P | . |
| A/CK/Indonesia/R60/05 | 2.1 | nd | + | S | . | L | . | D | P | . |

In this study, we have reported the protective potential of two mAb specific for hemagglutinin of a Z-genotype H5N1 virus A/Vietnam/1203/04. Micro-neutralization assays using A/Vietnam/1203/04 and A/Hong Kong/213/03, designed to select for antibodies not dependant on the 150s loop for binding, identified two mAb (VN04-2 and VN04-3) which showed similar levels of neutralization against both H5N1 viruses. The sequences of these monoclonal antibodies are listed below.

Since the ultimate goal is to, identify antibodies which can be used as passive antibody prophylaxis against human infection with H5N1 virus, we humanized the antibody prior to efficacy studies by replacing the constant regions of the mouse mAb with those of human IgG1 heavy chain and kappa light chain. The chimeric antibodies retained the specificity of the parent mouse mAbs. Humanization is a complex issue, designed to overcome the problems associated with use of mouse mAbs in humans, such as human anti-mouse-antibody (HAMA) responses that limits their repeated use: the extent of humanization versus the reduction of HAMA response is still an open question. Some human anti-chimeric antibody responses have been observed, however some human antibodies against more completely humanized mouse mAb have also been observed. Nevertheless, antibodies humanized by both methods used therapeutically and repetitively in the same individual are evident and the question of which is the best method still remains open. Perhaps the ultimate test will be in phase 1 clinical trials where the development of human antibodies against the introduced humanized antibody can be experimentally determined.

As a prelude to the possibility of testing the clinical application of these antibodies in human trials, their efficacy in an animal model needs to be determined. To this end, protection against lethal virus infection in a C57BL/6 mouse model both in a prophylactic and therapeutic context was performed. VN04-2-huG1 performed better in vivo than VN04-3-huG1 as complete protection against virus challenge with very little sign of disease with the lowest tested antibody concentration of 1 mg/kg was observed. This level of VN04-2-huG1 was not as effective therapeutically when used one or three days after infection, as protection was reduced to 80% and significant signs of disease were evident. However increasing the dosage of the antibody did restore complete protection and limit illness. As expected, a correlation of antibody dosage required for effective treatment versus the time of treatment after infection was evident, as more antibody was required to achieve similar therapeutic efficacy when the antibodies were introduced three days after infection compared to one day post infection. This result may allow for extension of the antibodies therapeutic potential to three days post infection. The efficacy of VN04-2-huG1 both as prophylaxis and therapy suggests that this antibody should be considered for further evaluation as a passive antibody prophylaxis against H5N1 virus infection for use in humans.

A potential drawback to the use of passive antibodies is the current high cost of large scale antibody production. This raises the costs of treatments utilizing antibodies, such as for RSV infection and autoimmune disease, to several thousands of dollars per treatment. It is also worthy of note that these antibodies are among the first commercially available antibodies for clinical use, a factor which contributes to the high cost and also that the amount of antibody administered is very high. Should an influenza pandemic arise, the increased burden on infrastructure as well as the likely effect on tourism and international trade would have a large impact on the economies of many countries. Therefore, the widespread use of protective antibodies to mitigate virus spread would become a governmental decision, as faster economic recovery through decisive and rapid containment of a pandemic would make the cost of the antibodies negligible when compared to the cost of a prolonged pandemic. However, antibody dosage for every member of the population may not be necessary. Recent studies examining containment strategies for pandemic influenza using a combination of geographically targeted prophylaxis with antiviral drugs and social distancing have suggested that a stockpile of 3 million doses of antiviral may be sufficient to contain an emerging pandemic at the source (Ferguson et al. Nature 2005, 437:209-214 and Longini et al. Science 2005, 309:1083-1087). Given the efficacy of VN04-2-huG1, it would not be unwarranted to suggest that passive antibody therapy with these antibodies could be used in a similar manner to antiviral drugs in this model, drastically reducing the amount of antibody needed for stockpiling to an achievable level.

While the antibodies described here are specific for the hemagglutinin of H5N1 viruses of the Z-genotype circulating in 2003/2004, the HI data presented here and that detailed in the abovementioned study identified the 140s antigenic loop as responsible for antibody binding and suggests a requirement for lysine at position 140. Hence the antibodies should be effective for all influenza viruses where there is a lysine at or around position 140 of the Hemagglutinin protein. However it should be noted that all of the HI assay negative strains contain a mutation at residue 94. While this residue faces inside the hemagglutinin trimer and would not be exposed for antibody binding, it may still have an effect on the performance of the HI assays, as was the case with mutation of a serine at position 223 to asparagine (Hoffmann et al. Proc Natl Acad Sci USA 2005, 102:12915-12920, raising the possibility that the negative result for the HI assays may be more a limitation of the assay rather then the ability of the antibody to bind to the virus.

A panel of proven protective antibodies is established against multiple 140s antigenic loop variants in accordance with the invention described herein. It is shown here the ‘proof of principle’ that passive antibody therapy is an effective tool for both prophylaxis against—and treatment of—highly pathogenic H5N1 influenza virus, providing the immediate immunity needed which combined with social distancing could limit the transmission of H5N1 virus to others and contain a future influenza pandemic.

Sequence of the mAb variable regions used to
construct VN04-2-HuG1 and VN04-3-HuG1

Nucleotide Sequence of the variable regions for
mAb VN04-2
VN04-2-VH (heavy chain)
SEQ ID No.1:
CAGGTGCAGCTGCAGGAGTCAGGACCTGACCTGGTGAAGCCTGGGGCTTC
AGTGAAGATGTCCTGCAAGGCTTCTGGTTACTCATTCACTTACTACCACA
TGCACTGGGTAAAGCAGACCCATGGAAAGAGCCTTGAGTGGATTGGACGT
ATTTATCCTTACAATGGTGGCACTAACTACAGCCAGAGGTTCAAGGGCAA
GGCCGTATTTACTGTGGACATGTCATCCAGCACAGCCTACATGGAGCTCC
GCAGCCTGACATCTGAGGACTCTGCGGTCTATTACTGTGCCAGAGATGCT
AACTACGAGGGGGACTGGTACTTCGATGTCTGGGGCCAAGGGACCACGGT
CACCGTCTCCTCA
VN04-2-VL (light chain)
SEQ ID No. 2
GACATCCAGATGACGCAGTCTCCATCCTCCTTTTCTGTATCTCTAGGAGA
CAGAGTCACCATTACTTGCAAGGCAAGTGAGGACATATATAATCGGTTAG
CCTGGTATCAGCAGAAACCAGGAAATGCTCCTAGGCTCTTAATATCTGAT
ACAACCAATTTGTGGATTGGGGTTCCTTCAAGATTCAGTGGCAGTGGATC
TGGAAAGGATTACACTCTCAGCATTACCAGTCTTCAGAGTGAAGATGTTG
CTACTTATTACTGTCAACAGTATTGGAGTTCTCGGACGTTCGGTGGAGGC
ACCAAGCTGGAAATCAAACGG
Nucleotide Sequence of the variable regions for
mAb VN04-3
VN04-3-VH (heavy chain)
SEQ ID No. 3
CAGGTGCAGCTGCAGGAGTCTGGACCTGACCTGGTGAAGCCTGGGGCTTC
AGTGATGATATCCTGCAAGGCTTCTGGTTACTCATTCACTTACTACTACA
TGCACTGGGTGAAGCAGAGCCATGGAAAGACCCTTGAGTGGATTGGACGT
ATTTATCCTTACAATGGTGATACTAACTACAACCGGAGGTTCAAGGACAA
GGCCATATTAACTGTAGACGAGTCATCCAGCACAGCCTACATGGAGCTCC
GCAGCCTGACATCTGAGGACTCTGCGGTCTATTTCTGTGCAAGAGATGCT
AACTACGAGGGGGACTGGTACTTCGATGTCTGGGGCCAAGGGACCACGGT
CACCGTCTCCTCA
VN04-3-VL (light chain)
SEQ ID No. 4
GACATCCAGATGACTCAGTCTCCATCCTCCTTTTCTGTATCTCTGGGAGA
CAGAGTGACCATTACTTGTAAGGCAAGTGAGGACATATATAATCGGGTAG
CCTGGTATCAGCAGAAACCAGGAAATGCTCCTAGGCTCTTAATGTCTGAT
GCAACCAGTTTGGGAGCTGGGGTTCCTTCAAGATTCAGTGGCAGTGGATC
TGGAAAGGATCACACTCTCAGCATTACCAGTCTTCAGACTGAAGATGTTG
CTACTTATTACTGTCAACAATATTGGAATACTCGGACGTTCGGTGGAGGC
ACCAAGCTGGAATCAAACGG
Amino acid Sequence of the Variable regions for
mAb VN04-2
VN04-2-VH
SEQ ID No. 5:
Gln Val Gln Leu Gln Glu Ser Gly Pro Asp Leu Val
Lys Pro Gly Ala Ser Val Lys Met Ser Cys Lys Ala
Ser Gly Tyr Ser Phe Thr Tyr Tyr His Met His Trp
Val Lys Gln Thr His Gly Lys Ser Leu Glu Trp Ile
Gly Arg Ile Pro Tyr Asn Gly Gly Thr Asn Tyr Ser
Gln Arg Phe Lys Gly Lys Ala Val Phe Thr Val Asp
Met Ser Ser Ser Thr Ala Tyr Met Glu Leu Arg Ser
Leu Thr Ser Glu Asp Ser Ala Val Tyr Tyr Cys Ala
Arg Asp Ala Asn Tyr Glu Gly Asp Trp Tyr Phe Asp
Val Trp Gly Gln Gly Thr Thr Val Thr Val Ser Ser
VN04-2-VL
SEQ ID No. 6
Asp Ile Gln Met Thr Gln Ser Pro Ser Ser Phe Ser
Val Ser Leu Gly Asp Arg Val Thr Ile Thr Cys Lys
Ala Ser Glu Asp Ile Tyr Asn Arg Leu Ala Trp Tyr
Gln Gln Lys Pro Gly Asn Ala Pro Arg Leu Leu Ile
Ser Asp Thr Thr Asn Leu Trp Ile Gly Val Pro Ser
Arg Phe Ser Gly Ser Gly Ser Gly Lys Asp Tyr Thr
Leu Ser Ile Thr Ser Leu Gln Ser Glu Asp Val Ala
Thr Tyr Tyr Cys Gln Gln Tyr Trp Ser Ser Arg Thr
Phe Gly Gly Gly Thr Lys Leu Glu Ile Lys Arg
Amino Acid Sequence of the variable regions for
mAb VN04-3
VN04-3-VH
SEQ ID No. 7
Gln Val Gln Leu Gln Glu Ser Gly Pro Asp Leu Val
Lys Pro Gly Ala Ser Val Met Ile Ser Cys Lys Ala
Ser Gly Tyr Ser Phe Thr Tyr Tyr Tyr Met His Trp
Val Lys Gln Ser His Gly Lys Thr Leu Glu Trp Ile
Gly Arg Ile Tyr Pro Tyr Asn Gly Asp Thr Asn Tyr
Asn Arg Arg Phe Lys Asp Lys Ala Ile Leu Thr Val
Asp Glu Ser Ser Ser Thr Ala Tyr Met Glu Leu Arg
Ser Leu Thr Ser Glu Asp Ser Ala Val Tyr Phe Cys
Ala Arg Asp Ala Asn Tyr Glu Gly Asp Trp Tyr Phe
Asp Val Trp Gly Gln Gly Thr Thr Val Thr Val Ser
Ser
VN04-3-VL
SEQ ID No. 8
Asp Ile Gln Met Thr Gln Ser Pro Ser Ser Phe Ser
Val Ser Leu Gly Asp Arg Val Thr Ile Thr Cys Lys
Ala Ser Glu Asp Ile Tyr Asn Arg Val Ala Trp Tyr
Gln Gln Lys Pro Gly Asn Ala Pro Arg Leu Leu Met
Ser Asp Ala Thr Ser Leu Gly Ala Gly Val Pro Ser
Arg Phe Ser Gly Ser Gly Ser Gly Lys Asp His Thr
Leu Ser Ile Thr Ser Leu Gln Thr Glu Asp Val Ala
Thr Tyr Tyr Cys Gln Gln Tyr Trp Asn Thr Arg Thr
Phe Gly Gly Gly Thr Lys Leu Glu Ile Lys Arg

We claims:

1. An antibody that specifically binds to a 140s protruding loop of a hemagglutinin protein wherein the hemagglutinin is a H5 of an influenza A virus.

2. The antibody of claim 1 wherein said antibody is an immunoglobulin comprising an immunoglobulin heavy chain comprising a variable domain comprising an amino acid sequence selected from the group consisting of SEQ ID NOS: 5 and 7.

3. The antibody of claim 1 wherein said antibody is an immunoglobulin comprising an immunoglobulin light chain comprising a variable domain comprising an amino acid sequence selected from the group consisting of SEQ ID NOS: 6 and 8.

4. The antibody of claim 2 or 3 wherein the immunoglobulin is an IgG1 kappa immunoglobulin.

5. The antibody of claim 4 wherein the immunoglobulin comprises a human IgG1 constant region within a heavy chain of the immunoglobulin and a human constant region within a light chain of the immunoglobulin.

6. The antibody of claim 5 wherein the immunoglobulin comprises fully or partially human framework regions within the variable domain of said heavy chain and within the variable domain of said light chain.

7. The antibody of claim 5 wherein the immunoglobulin comprises murine framework regions within the variable domain of the heavy chain and within the light chain.

8. The antibody of claim 1 wherein the hemagglutinin is derived from type A influenza strain H5N1 or Z-genotype of type A influenza strain H5N1.

9. The antibody of claim 8 wherein the 140s protruding loop comprises a glutamine at position 138 or a lysine at position 140 or a serine at position 141 of the protein.

10. An Antibody that specifically binds to the 140s protruding loop of hemagglutinin protein wherein the hemagglutinin is a H5 of an influenza A virus, comprising an immunoglobulin variable heavy (VH) domain selected from the group consisting of:
(a) a polypeptide comprising the amino acid sequence of SEQ ID NO: 5 or 7;
(b) a polypeptide having at least 90% sequence identity to the amino acid sequence of SEQ ID NO: 5 or 7, wherein said immunoglobulin specifically binds to the 140s protruding loop of hemagglutinin;
(c) a polypeptide that is encoded by the nucleotide sequence of SEQ ID NO: 1 or 3; or
(d) a polypeptide that is encoded by a nucleotide sequence that is at least 90% identical to the nucleotide sequence of SEQ ID NO: 1 or 3, wherein said immunoglobulin specifically binds to the 140s protruding loop of hemagglutin.

11. The antibody of claim 10 further comprising an immunoglobulin light chain comprising a variable domain selected from the group consisting of:
(a) a polypeptide comprising the amino acid sequence of SEQ ID NO: 6 or 8;
(b) a polypeptide having at least 90% sequence identity to the amino acid sequence of SEQ ID NO: 6 or 8, wherein said immunoglobulin specifically binds to the 140s protruding loop of hemagglutinin;
(c) a polypeptide that is encoded by the nucleotide sequence of SEQ ID NO: 2 or 4; or
(d) a polypeptide that is encoded by a nucleotide sequence that is at least 90% identical to the nucleotide sequence of SEQ ID NO: 2 or 4, wherein said immunoglobulin specifically binds to the 140s protruding loop of hemagglutin.

12. The antibody of any one of claims 1 to 11, wherein the antibody is conjugated to an agent selected from the group consisting of a therapeutic agent, a prodrug, a peptide, a protein, an enzyme, a virus, a lipid, a biological response modifier, a pharmaceutical agent, and PEG.

13. A composition comprising the antibody of any one of claims 1 to 12, and a carrier.

14. The composition of claim 13 for use in preventing or treating influenza virus infection in a subject comprising administering to said subject an effective amount of a composition.

15. The composition of claim 14 wherein the influenza virus infection is a type A influenza strain H5N1 or Z-genotype of type A influenza strain H5N1.

16. An isolated polynucleotide comprising a nucleic acid encoding a variable heavy (VH) domain of an immunoglobulin heavy chain, wherein said VH domain is selected from the group consisting of:
(a) a nucleic acid molecule comprising the nucleotide sequence of SEQ ID NO: 1 or 3;
(b) a nucleic acid molecule comprising a nucleotide sequence having at least 90% sequence identity to the nucleotide sequence of SEQ ID NO: 1 or 3, wherein said immunoglobulin specifically binds to the 140s protruding loop of hemagglutinin protein wherein the hemagglutinin is a H5 of an Influenza A virus;
(c) a nucleic acid that encodes a polypeptide comprising the amino acid sequence of SEQ ID NO: 5 or 7; or
(d) a nucleic acid molecule that encodes a polypeptide that is at least 90% identical to the amino acid sequence of SEQ ID NO: 5 or 7, wherein said immunoglobulin specifically binds to the 140s protruding loop of hemagglutinin protein wherein the hemagglutinin is a H5 of an Influenza A virus.

17. An isolated polynucleotide comprising a nucleic acid encoding a variable light (VL) domain of an immunoglobulin light chain, wherein said VL domain is selected from the group consisting of:
(a) a nucleic acid molecule comprising the nucleotide sequence of SEQ ID NO: 2 or 4;
(b) a nucleic acid molecule comprising a nucleotide sequence having at least 90% sequence identity to the nucleotide sequence of SEQ ID NO: 2 or 4, wherein said immunoglobulin specifically binds to the 140s protruding loop of hemagglutinin protein wherein the hemagglutinin is a H5 of an Influenza A virus;
(c) a nucleic acid that encodes a polypeptide comprising the amino acid sequence of SEQ ID NO: 6 or 8; or
(d) a nucleic acid molecule that encodes a polypeptide that is at least 90% identical to the amino acid sequence of SEQ ID NO: 6 or 8, wherein said immunoglobulin specifically binds to the 140s protruding loop of hemagglutinin protein wherein the hemagglutinin is a H5 of an Influenza A virus.

18. A method for preventing or treating influenza virus infection in a subject comprising administering to said subject an effective amount of a composition comprising the antibody according to any one of claims 1 to 12.

19. The method of claim 18, wherein said influenza virus comprises a type A influenza strain H5N1 or a Z-genotype of type A influenza strain H5N1.

20. A pharmaceutical composition comprising the antibody according to any one of claims 1 to 12.

21. A method of diagnosing infection with an influenza virus in a subject comprising contacting a sample from the subject with the antibody according to any one of claims 1 to 12 and detecting the presence of hemagglutinin wherein the hemagglutinin is a H5 of an influenza A virus.

22. A kit for detecting the presence of a hemagglutinin antigen wherein the hemagglutinin is a H5 of an influenza A virus comprising:
(a) the antibody according to any one of claims 1 to 12; and
(b) instructions for detecting influenza A virus.

23. The kit of claim 22 wherein the influenza A virus comprises, a type A influenza strain H5N1 or a Z-genotype of type A influenza strain H5N1 further comprising:
(a) a substrate capable of producing a detectable product when in contact with a complex of the antibody and the hemagglutinin antigen.
